(19) United States
(12) Patent Application Publication
(10) Pub. No.: US 2024/0277224 A1
(43) Pub. Date: Aug. 22, 2024

(54) OPTICAL COHERENCE TOMOGRAPHY (OCT) SELF-TESTING SYSTEM, OPTICAL COHERENCE TOMOGRAPHY METHOD, AND EYE DISEASE MONITORING SYSTEM

(71) Applicant: Medimaging Integrated Solution, Inc., Hsinchu (TW)

(72) Inventors: Chu-Ming Cheng, Hsinchu (TW); Wei Ting Tseng, Hsinchu (TW); LI-REN CAI, Hsinchu (TW); Hung-Chin Chen, Hsinchu (TW); CHIEN-CHI HUANG, Hsinchu (TW); Yung-En Kuo, Hsinchu (TW); PEI-SHENG WU, Hsinchu (TW)

(21) Appl. No.: 18/540,661

(22) Filed: Dec. 14, 2023

(30) Foreign Application Priority Data
Feb. 21, 2023 (TW) 112106194

(51) Int. Cl. A61B 3/10; A61B 3/00; A61B 3/12; A61B 3/14; G06T 5/40; G06T 5/70; G06T 7/13

(52) U.S. Cl. CPC A61B 3/102; A61B 3/0058; A61B 3/1225; A61B 3/14; G06T 5/40; G06T 5/70; G06T 7/13; G06T 2207/10101; G06T 2207/30041

(57) ABSTRACT

The invention provides an optical coherence tomography self-testing system, an optical coherence tomography method and an ocular disease monitoring system. The optical coherence tomography self-testing system comprises a camera device, an external display module and a communication module. The camera device includes an image-capturing module and a processing module. The image-capturing module captures a plurality of ocular images. The processing module is connected to the image-capturing module, and the processing module determines whether a position offset value between the pupil center position of a tested eyeball and an optical axis of the image-capturing module is within a preset error range. If the position offset value is within the preset error range, the plurality of ocular images is stored as a plurality of displayed images. The external display module displays one of the plurality of displayed images and a status light after the image-capturing module has completed image capturing.

### BACKGROUND OF THE INVENTION

### 1. Field of the Invention

The present invention provides an optical scanning device and a monitoring system with an optical scanning device, particularly to an optical coherence tomography (OCT) self-testing system, an OTC method, and an ocular disease monitoring system, which enable a user to inspect whether his retina is diseased.

### 2. Description of the Prior Art

The optical coherence tomography (OTC) device has been an important inspection instrument in ophthalmology. The OTC device enables an ophthalmologist to watch the layered structure of the retina of a patient in real time, greatly favoring the diagnosis of ocular diseases. However, the traditional OCT device is bulky and expensive. The patients have to go to the hospital for OCT inspection. The physicians have to view the results of OCT inspection via the image system or high-end display device on-site. The OCT device is often used to inspect glaucoma, macular degeneration, and diabetic retinopathy.

The age-related macular degeneration is caused by aging, deterioration of ocular functions, or other risk factors, such as age, smoking, high myopia, and air pollution. Macular degeneration normally occurs in the people aged over 50. However, the patients are hard to perceive macular degeneration in the early stage. The early-stage macular degeneration cannot be detected unless the patient is monitored with optical devices for a long period of time. While macular degeneration becomes serious, the patient may experience visual field variation, defective color vision, and impaired vision. In fact, the patients of macular degeneration rarely seek for medical care until the visual field has been seriously damaged in the late stage.

Retinopathy needs monitoring for a long period of time. Diabetic retinopathy is a major ophthalmological disease for the elder people. This is because hyperglycemia raises the aggregation of platelets. Platelet aggregation may induce microvascular clogging or hypoxia in retina and thus lead to abnormal angiogenesis. Although controlling blood sugar is helpful for preventing retinopathy, a longer disease progress may still make the patient suffer from retinopathy. Retinopathy is a disease slowly exacerbated with time. Thus, the patient must frequently go to the hospital for inspection, which should consume much time and labor for both the patient and hospital.

### SUMMARY OF THE INVENTION

One objective of the present invention is to provide an optical coherence tomography (OCT) self-testing system, an OTC method, and an ocular disease monitoring system, which enable a patient to perform inspection by himself without troublesome and complicated operations, whereby the patient may be more willing to monitor his ocular diseases persistently.

The present invention proposes an optical coherence tomography (OCT) self-testing system, which comprises a camera device, an external display module, and a communication module. The camera device includes an image-capturing module and a processing module. The image-capturing module captures a plurality of ocular images. The processing module is connected with the image-capturing module, determining whether the position offset value between the center of the pupil of the tested eyeball and the optical axis of the image-capturing module is within a preset error range. If the position offset value is within the preset error range, the processing module determines whether the position offset value is unchanged within a first preset time interval. If the position offset value is unchanged within the first preset time interval, the processing module stores the plurality of ocular images, which is captured within the first time interval, as a plurality of displayed images. The external display module is coupled to the processing module and displays one of the plurality of displayed images and a status light, wherein the status light indicates whether the image-capturing module has completed image capturing. The communication module is connected with the processing module and transmits the plurality of ocular images to the exterior.

The present invention also proposes an optical coherence tomography method, which is applied to an optical coherence tomography self-testing system. The optical coherence tomography self-testing system comprises a camera device, an external display module, and a communication module. The camera device includes an image-capturing module and a processing module. The optical coherence tomography method uses the optical coherence tomography self-testing system to undertake the following steps: using the processing module to determine whether the position offset value between the center of the pupil of the tested eyeball and the optical axis of the image-capturing module is within a preset error range; if the position offset value is within the preset error range, using the processing module to determine whether the position offset value is unchanged within a first preset time interval; if the position offset value is unchanged within the first preset time interval, using the image-capturing module to capture a plurality of ocular images, and storing the plurality of ocular images, which is captured within the first time interval, as a plurality of displayed images; after the image-capturing module has completed image capturing, using the external display module to display one of the plurality of displayed images and a status light, wherein the status light indicates whether the image-capturing module has completed image capturing; and using the communication module to transmit the plurality of ocular images to the exterior.

The present invention also proposes an ocular disease monitoring system, which comprises an optical coherence tomography self-testing system and a computation system. The optical coherence tomography self-testing system comprises a camera device, an external display module, and a communication module. The camera device includes an image-capturing module and a processing module. The image-capturing module captures a plurality of ocular images. The processing module is connected with the image-capturing module, determining whether the position offset value between the center of the pupil of the tested eyeball and the optical axis of the image-capturing module is within a preset error range. If the position offset value is within the preset error range, the processing module determines whether the position offset value is unchanged within a first preset time interval. If the position offset value is unchanged within the first preset time interval, the processing module stores the plurality of ocular images, which is captured within the first time interval, as a plurality of displayed images. The external display module is coupled to the processing module and displays one of the plurality of displayed images and a status light, wherein the status light indicates whether the image-capturing module has completed image capturing. The communication module is connected with the processing module and transmits the plurality of ocular images to the exterior. The computation system is in signal communication with the optical coherence tomography self-testing system, receiving the plurality of ocular images, and inspecting the plurality of ocular images to generate an inspection result, and transmitting the inspection result to the optical coherence tomography self-testing system.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a block diagram of an optical coherence tomography self-testing system according to one embodiment of the present invention;

FIG. 2 is a diagram schematically showing an image-capturing module of an optical coherence tomography self-testing system according to one embodiment of the present invention;

FIGS. 3(A)-3(D) schematically show images presented by an internal display module according to one embodiment of the present invention;

FIG. 4 is a first flowchart of an OCT method according to one embodiment of the present invention;

FIG. 5 is a second flowchart of an OCT method according to one embodiment of the present invention;

FIG. 6 is a flowchart of a pre-treatment process of an OCT method according to one embodiment of the present invention;

FIG. 7 is a third flowchart of an OCT method according to one embodiment of the present invention;

FIG. 8 is a fourth flowchart of an OCT method according to one embodiment of the present invention;

FIG. 9 is a fifth flowchart of an OCT method according to one embodiment of the present invention; and

FIG. 10 is a block diagram schematically showing an ocular disease monitoring system according to one embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The embodiments of the present invention will be demonstrated thereinafter in cooperation with the attached drawings.

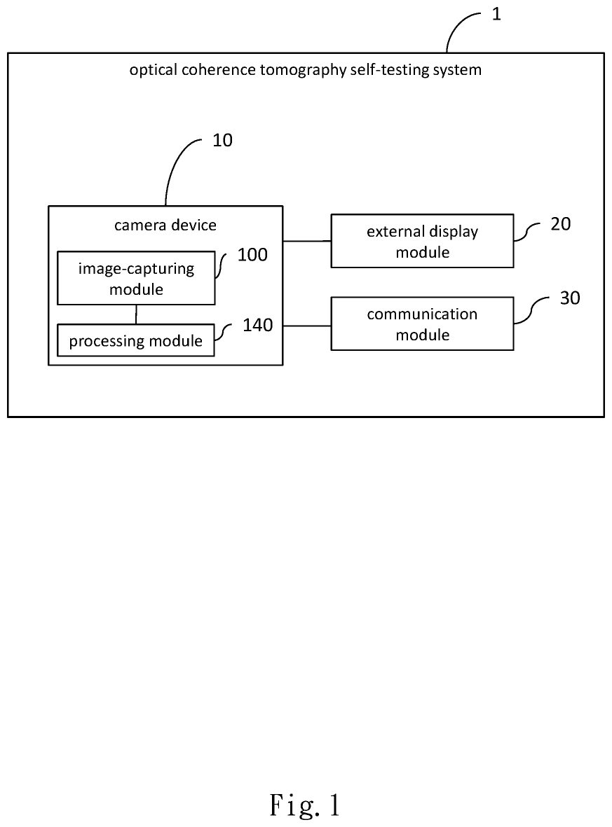

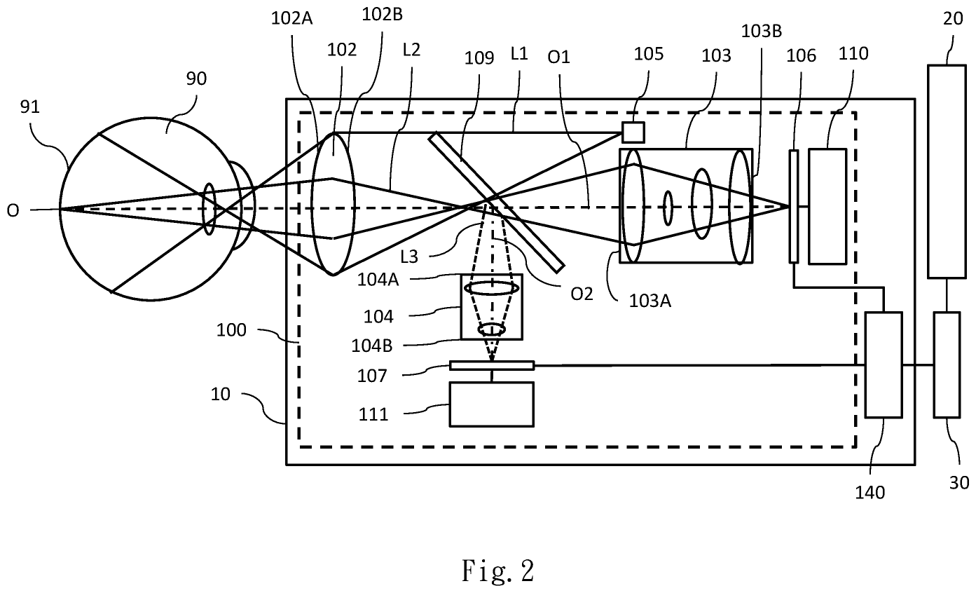

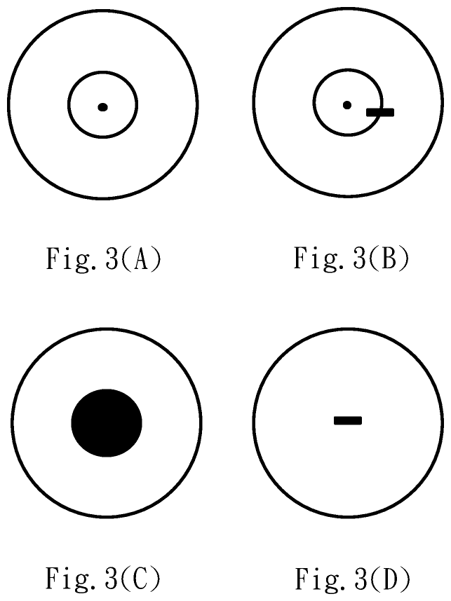

Refer to FIG. 1, FIG. 2, and FIGS. 3(A)-3(D). FIG. 1 is a block diagram of an optical coherence tomography self-testing system according to one embodiment of the present invention. FIG. 2 is a diagram schematically showing an image-capturing module of an optical coherence tomography self-testing system according to one embodiment of the present invention. FIGS. 3(A)-3(D) schematically show images presented by an internal display module according to one embodiment of the present invention.

The optical coherence tomography (OCT) self-testing system 1 of the present invention comprises a camera device 10, an external display module 20, and a communication module 30.

The camera device 10 includes an image-capturing module 100 and a processing module 140. The image-capturing module 100 captures a plurality of ocular images. The processing module 140 is connected with the image-capturing module 100, determining whether the position offset value between the center of the pupil of a tested eyeball 90 and the optical axis O of the image-capturing module 100 is within a preset error range. If the position offset value is outside the preset error range, the processing module 140 performs determination once again. If the position offset value is within the preset error range, the processing module 140 determines whether the position offset value is unchanged within a first preset time interval. If the position offset value is changed within the first preset time interval, the processing module 140 calculates the position offset value once again after a second time interval. If the position offset value is unchanged within the first preset time interval, the processing module 140 stores the plurality of ocular images, which is captured within the first time interval, as a plurality of displayed images. According to a preset tracking rule and a preset focusing rule, the processing module 140 analyzes a relative position of the optical axis O of the image-capturing module 100 and the center of the pupil of the tested eyeball 90 to generate the plurality of displayed images.

The external display module 20 is coupled to the processing module 140. After the processing module 140 has completed image capturing, the external display module 20 displays one of the plurality of displayed images and a status light, wherein the status light indicates the status of the image capturing performed by the image-capturing module. The communication module 30 is connected with the processing module 140 and transmits the plurality of ocular images to the exterior.

The image-capturing module 100 includes a first lens assembly 102, a second lens assembly 103, a third lens assembly 104, an illumination element 105, a sensing module 106, a splitter 109, an internal display module 107, a first focal-length regulator 110, and a second focal-length regulator 111.

The first lens assembly 102 has a first-lens first side 102A, and a first-lens second side 102B, which are opposite to each other. The first-lens first side 102A faces the tested eyeball 90 of a testee. The second lens assembly 103 has a second-lens first side 103A, and a second-lens second side 103B, which are opposite to each other. The second-lens first side 103A faces the first-lens second side 102B. The second lens assembly 103 is disposed coaxially with the first lens assembly 102 at the first-lens second side 102B. The second lens assembly 103 includes at least one liquid lens. The illumination element 105 is disposed at the first-lens second side 102B. The illumination element 105 generates a light beam L1 to illuminate the external region of the eyeball 90 of the testee. The light beam L1 is focused at a fundus 91 of the eyeball 90 by the first lens assembly 102. The illumination element 105 may generate visible light or infrared light to function as the light beam L1 for photographing the external region of the eyeball 90.

The splitter 109 is disposed at a position between the first-lens second side 102B and the second-lens first side 103A. The splitter 109 splits the optical axis O passing through the first lens assembly 102 into a first optical path O1 and a second optical path O2, and the first optical path O1 is the extension of the optical axis O of the first lens assembly 102. The sensing module 106 is connected with the processing module 140 and disposed at the second-lens second side 103B. An imaging light beam L2 from the tested eyeball 90 is focused by the first lens assembly 102 and the second lens assembly 103 to form images on the sensing module 106. The sensing module 106 receives the imaging light to form a plurality of ocular images. The third lens assembly 104 is coaxially disposed at the second optical path O2, having a third-lens first side 104A, and a third-lens second side 104B, which are opposite to each other. The third-lens first side 104A faces the splitter 109. The splitter 109 is optically coupled to the internal display module 107 and the second lens assembly 103.

The internal display module 107 is connected with the processing module 140 and disposed at the third-lens second side 104B. The processing module 140 transmits a plurality of displayed images to the internal display module 107. The internal display module 107 presents a plurality of displayed images each including a picture frame of the area of the captured image. The internal display module 107 generates an imaging light L3, which is corresponding to the plurality of the displayed images. The imaging light L3, which passes through the third lens assembly 104, the splitter 109 and the first lens assembly 102 in sequence, is focused to the tested eyeball 90.

The first focal-length regulator 110 is coupled to the processing module 140, driving the second lens assembly 103 to physically move along the first optical path O1, adjusting the curvature of at least one liquid lens, and thus modifying the focal length of the at least one liquid lens. The second focal-length regulator 111 is coupled to the processing module 140, driving the internal display module 107 to move along the second optical path O2 of the third lens assembly 104, or adjusting the position of the third lens assembly 104, and then making the imaging light form images on the fundus 91 of the tested eyeball 90.

Hence, the testee may adjust the relative position of the testee and the camera device 10 to realize automatic pupil alignment according to the reminding information presented by the internal display module 107.

Suppose that the position offset value of the position of the pupil of the testee and the optical axis O is d₁. While the position offset value in the XY direction dₓ_{₁}_{y}_{₁}≤0.2 mm, and the position offset value in the Z direction d_{z}_{₁}≤0.3 mm, the camera device 10 can determine that the position offset value d₁ is within the preset error range. It indicates that the eye of the testee is at a correct position. Then, the internal display module 107 presents information to remind the testee of staying still.

Next, the camera device 10 determines whether the position offset value d₁ is unchanged within a first preset time interval, such as 0.5 seconds. If the position offset value d₁ of the testee is unchanged within 0.5 seconds, the image-capturing module 100 uses the first focal-length regulator 110 and the second focal-length regulator 111 (such as a three-axis motor) to automatically fine tune the optical path to the optimized position for automatic photographing and then stores the ocular images captured within 0.5 seconds as the displayed images.

If the position offset value d1 is changed within the first preset time interval, the camera device 10 detects the position offset value once again after a second preset time interval, such 15 seconds. Suppose that the new position offset value is d₂. While the position offset value in the XY direction dₓ_{₂}_{y}_{₂}>0.2 mm, and the position offset value in the Z direction d_{z}_{₂}>0.3 mm, the camera device 10 can determine that the position offset value d₂ is outside the preset error range. It indicates that the distance between the pupil of the testee and the camera device 10 is too large or too small.

The internal display module 107 presents the picture frames of the areas of captured images, as shown in FIGS. 3(A)-3(D), wherein the outer circles are the picture frames of the areas of the captured images, which are the optimized areas to capture images of the testee eyeball 90 in the camera device 10.

While the internal display module 107 presents FIG. 3(A), it indicates that the OCT self-testing system 1 does not detect the pupil of the testee. While the internal display module 107 presents FIG. 3(B), it indicates that the distance between camera device 10 and the pupil of the testee is too large. While the internal display module 107 presents FIG. 3(C), it indicates that the pupil of the testee is too close to the camera device 10. While the internal display module 107 presents FIG. 3(C), it is a picture that the internal display module 107 presents to remind the testee of keeping still.

While the internal display module 107 presents the picture frame of the area of the captured image, the testee may adjust the relative position between the tested eyeball 90 and the camera device 10 according to the presented picture, whereby the testee may adjust the tested eyeball 90 to the optimized image-capturing area by himself.

If the testee moves while the processing module 140 begins to determine whether the testee keeps unchanged within the first preset time interval, the processing module 140 makes the image-capturing module 100 capture a plurality of ocular images once again after the second preset time interval has elapsed.

Suppose that the first preset time interval is 0.5 seconds and that the second preset time interval is 15 seconds. If the testee keeps stationary within 0.5 seconds, the images are captured automatically. If the testee moves within 0.5 seconds, the images are captured once again 15 seconds later.

If the testee remains stationary within the first preset time interval, the image-capturing module 100 may capture images automatically.

The status light may remind the testee of the medical-care requirement status and the measurement status of the OCT self-testing system 1. The status light may present different colors. For example, the status light may present four colors-blue, yellow, red and green. While the status light of the external display module 20 presents blue color, it indicates that the self-testing image capturing by the testee is successful and the image-capturing module has completed image capturing. While the status light of the external display module 20 presents yellow color, it indicates that image capturing fails. While the status light of the external display module 20 presents red color, it indicates that the OCT self-testing system 1 proposes that the testee needs to verify the related health conditions with medical personnel. While the status light of the external display module 20 presents green color, it indicates that the testee does not need medical treatment currently.

Therefore, the patient needn't go to a clinic or hospital for troublesome and complicated inspections but can perform tests by himself at home. The present invention can repeat photographing within a short time interval to increase the accuracy of self-test, not only encouraging patients to perform self-test but also providing information for the medical personnel to draw up precision treatment plans.

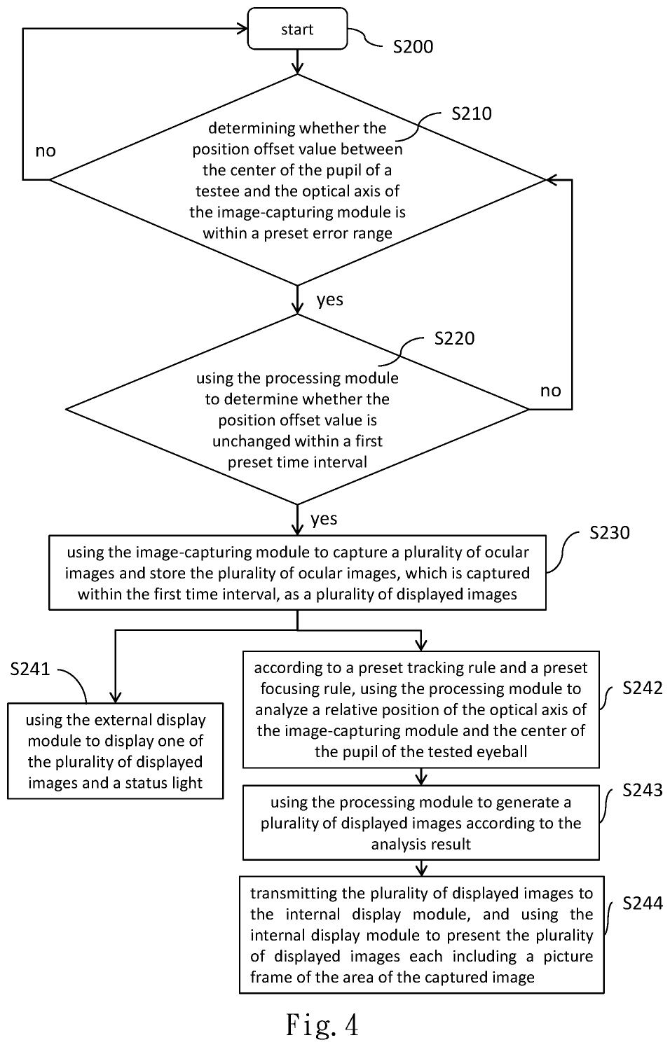

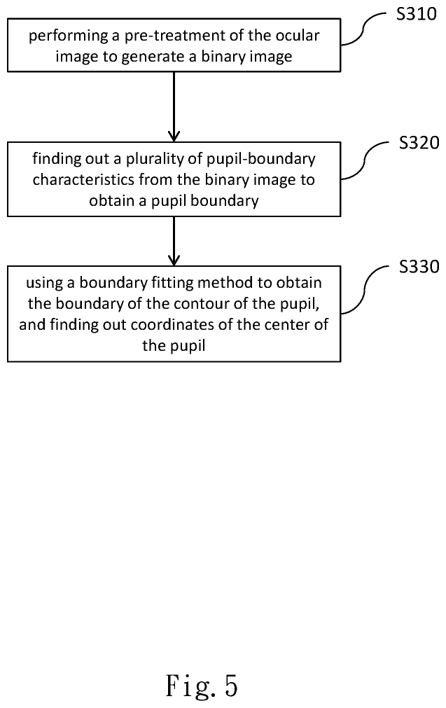

Refer to FIG. 4 and FIG. 5. FIG. 4 is a first flowchart of an OCT method according to one embodiment of the present invention. FIG. 5 is a second flowchart of an OCT method according to one embodiment of the present invention.

As shown in FIG. 4, the process starts from Step S200. Next, the process proceeds to Step S210: determining whether the position offset value between the center of the pupil of a testee and the optical axis of the image-capturing module is within a preset error range.

If the position offset value is within the error range, the process proceeds to Step S220: using the processing module to determine whether the position offset value is unchanged within a first preset time interval. If the position offset value is outside the error range, the process returns to Step S200.

If the position offset value is changed during Step S220, the process performs Step S210 after a second time interval to determine once again whether the position offset value is within the error range.

If the position offset value is unchanged within the first time interval, the process proceeds to Step S230: using the image-capturing module to capture a plurality of ocular images and store the plurality of ocular images, which is captured within the first time interval, as a plurality of displayed images.

After the ocular images have been captured, the process respectively proceeds to Step S241 and Step S242, wherein
Step S241: using the external display module to display one of the plurality of displayed images and a status light, wherein the status light can indicate whether the image-capturing module has completed image capturing;
Step S242: according to a preset tracking rule and a preset focusing rule, using the processing module to analyze a relative position of the optical axis O of the image-capturing module and the center of the pupil of the tested eyeball.

After Step S242, the process proceeds to Step S243: using the processing module to generate a plurality of displayed images according to the analysis result.

Then, the process proceeds to Step S244: transmitting the plurality of displayed images to the internal display module, and using the internal display module to present the plurality of displayed images each including a picture frame of the area of the captured image.

As shown in FIG. 5, Step S242, wherein the processing module analyzes the ocular images according to a preset tracking rule and a preset focusing rule, further comprises
Step S310: performing a pre-treatment of the ocular image to generate a binary image;
Step S320: finding out a plurality of pupil-boundary characteristics from the binary image to obtain a pupil boundary; and
Step S330: using a boundary fitting method to obtain the boundary of the contour of the pupil, and finding out coordinates of the center of the pupil,
   wherein the least square method is used to calculate the center of the fitted shape; the ellipse fitting method is used to calculate the sum of the squares of the distance from the center of the ellipse to the boundary to obtain the radius and the coordinates of the center, whereby the pupil can be tracked precisely; the processing module works out the position offset value between the tested eyeball and the optical axis of the second lens assembly according to the coordinates of the center of the pupil.

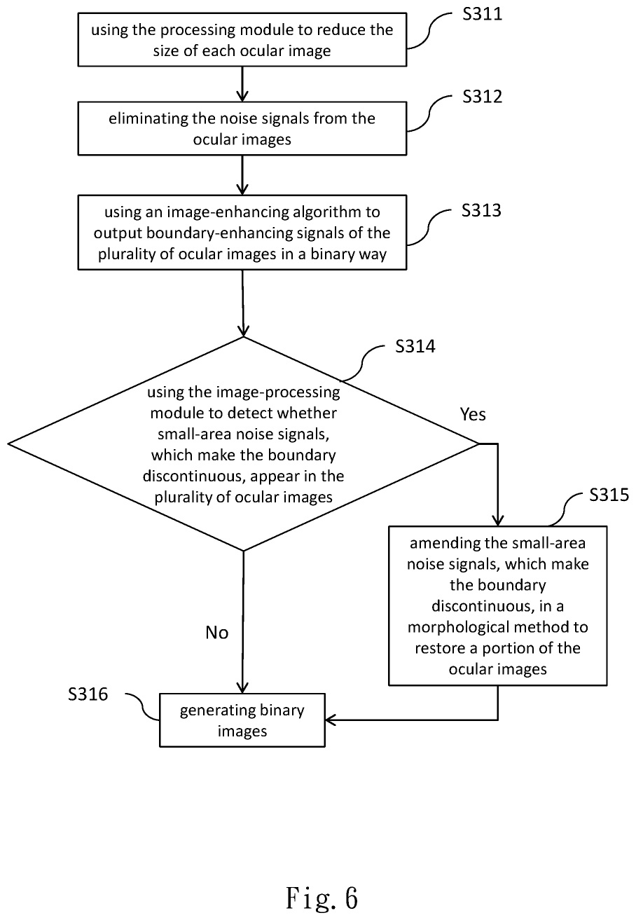

Refer to FIG. 6. FIG. 6 is a flowchart of a pre-treatment process of an OCT method according to one embodiment of the present invention.

As shown in FIG. 6, Step S310 in FIG. 5 further comprises Steps S311-S316, wherein signal processing and digital conversion of the ocular images will be undertaken.

Firstly is undertaken Step S311: using the processing module to reduce the size of each ocular image, whereby computing is accelerated;

Next, the process proceeds to Step S312: eliminating the noise signals from the ocular images, wherein a noise-eliminating algorithm is used to eliminate noise signals so as to increase the accuracy of the algorithm;

After the noise signals of the ocular images have been eliminated, the process proceeds to Step S313: using an image-enhancing algorithm to output boundary-enhancing signals of the plurality of ocular images in a binary way;

Next, the process proceeds to Step S314: using the image-processing module to detect whether small-area noise signals, which make the boundary discontinuous, appear in the plurality of ocular images;

If the small-area noise signals, which make the boundary discontinuous, appear in the plurality of ocular images, the process proceeds to Step S315: amending the small-area noise signals, which make the boundary discontinuous, in a morphological method to restore a portion of the ocular images.

Next, the process proceeds to Step S316: generating binary images.

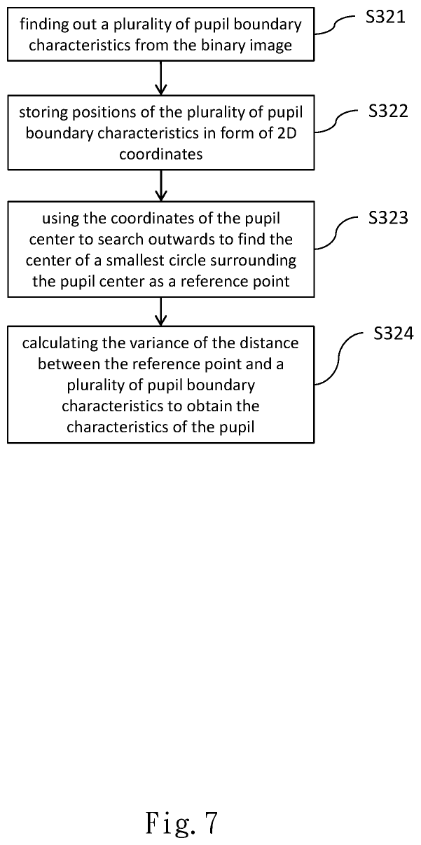

Refer to FIG. 7. FIG. 7 is a third flowchart of an OCT method according to one embodiment of the present invention.

As shown in FIG. 7, Step S320 in FIG. 5 further comprises Steps S321-S324, wherein
Step S321: finding out a plurality of pupil boundary characteristics from the binary image;
Step S322: storing positions of the plurality of pupil boundary characteristics in form of 2D coordinates;
Step S323: using the coordinates of the pupil center to search outwards to find the center of a smallest circle surrounding the pupil center as a reference point;
Step S324: calculating the variance of the distance between the reference point and a plurality of pupil boundary characteristics to obtain the characteristics of the pupil.

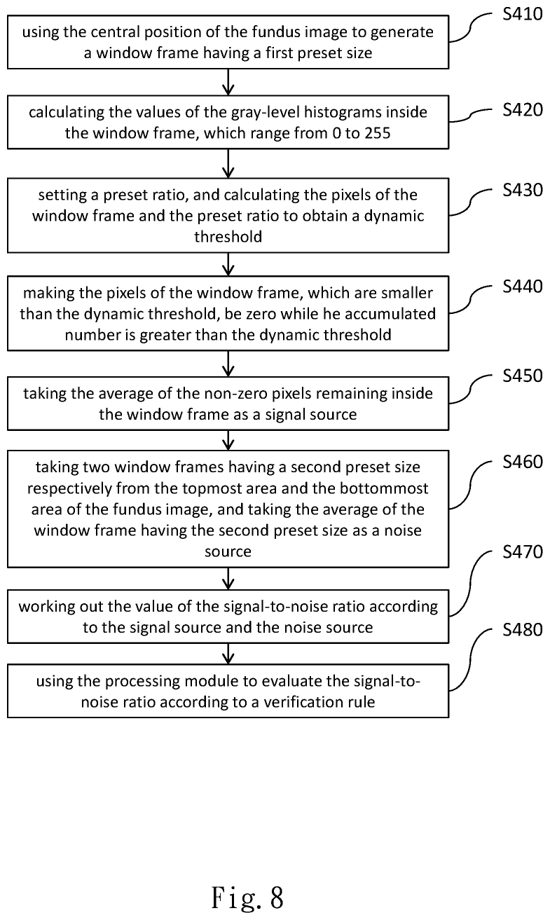

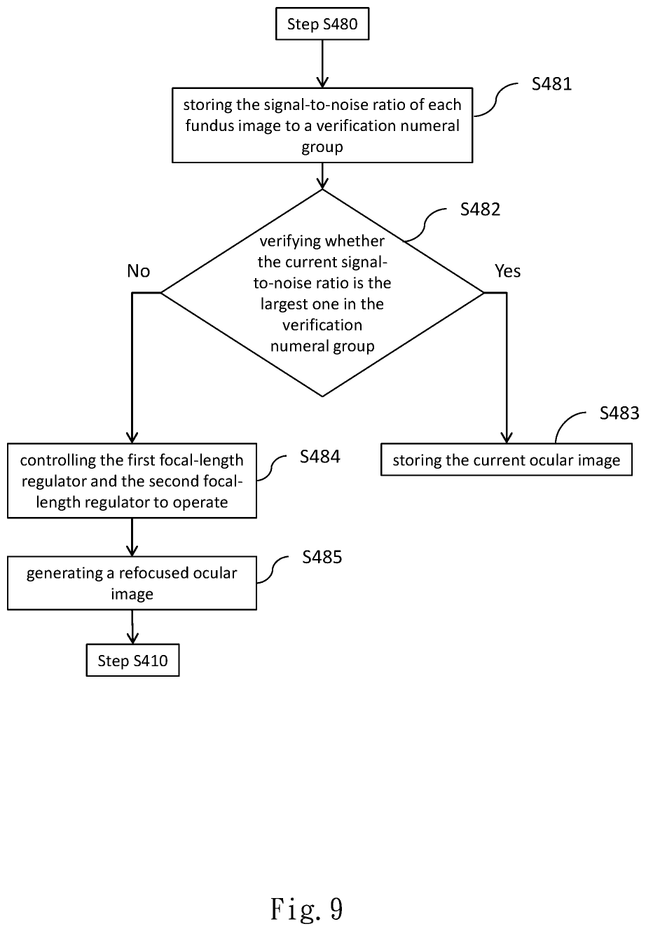

Refer to FIG. 8 and FIG. 9. FIG. 8 is a fourth flowchart of an OCT method according to one embodiment of the present invention. FIG. 9 is a fifth flowchart of an OCT method according to one embodiment of the present invention.

As shown in FIG. 8, Step S242 in FIG. 4, wherein the signal processing module analyzes each ocular image according to the preset focusing rule, further comprises Steps S410-S480, wherein
Step S410: using the central position of the ocular image to generate a window frame having a first preset size, wherein the first preset size may be 800×512;
Step S420: calculating the values of the gray-level histograms inside the window frame, which range from 0 to 255;
Step S430: setting a preset ratio, and calculating the pixels of the window frame and the preset ratio to obtain a dynamic threshold, wherein the better preset ratio is 0.9; the value generated by multiplying 0.9 and the value of the pixels of the window frame is the better dynamic threshold;
Step S440: making the pixels inside the window frame, which are smaller than the dynamic threshold, be zero while the accumulated number is greater than the dynamic threshold, whereby 90% image inside the window frame is set to be zero to eliminate too much noise;
Step S450: taking the average of the non-zero pixels remaining inside the window frame as a signal source;
Step S460: taking two window frames having a second preset size respectively from the topmost area and the bottommost area of the fundus image, and taking the average of the window frames having the second preset size as a noise source, wherein the second preset size may be 30×512;
Step S470: working out the value of the signal-to-noise ratio according to the signal source and the noise source; and
Step S480: using the processing module to evaluate the signal-to-noise ratio according to a verification rule.

As shown in FIG. 9, Step S480 further comprises
Step S481: storing the signal-to-noise ratio of each fundus image to a verification numeral group;
Step S482: verifying whether the current signal-to-noise ratio is the largest one in the verification numeral group;
Step S483: storing the current ocular image if the current signal-to-noise ratio is the largest one in the verification numeral group;
Step S484: controlling the first focal-length regulator and the second focal-length regulator once again to make the imaging light focused on the sensing module if the current signal-to-noise ratio is not the largest one in the verification numeral group;
Step S485: generating a refocused ocular image, and then returning to Step S410 for recalculating the signal-to-noise ratio of the current ocular image.

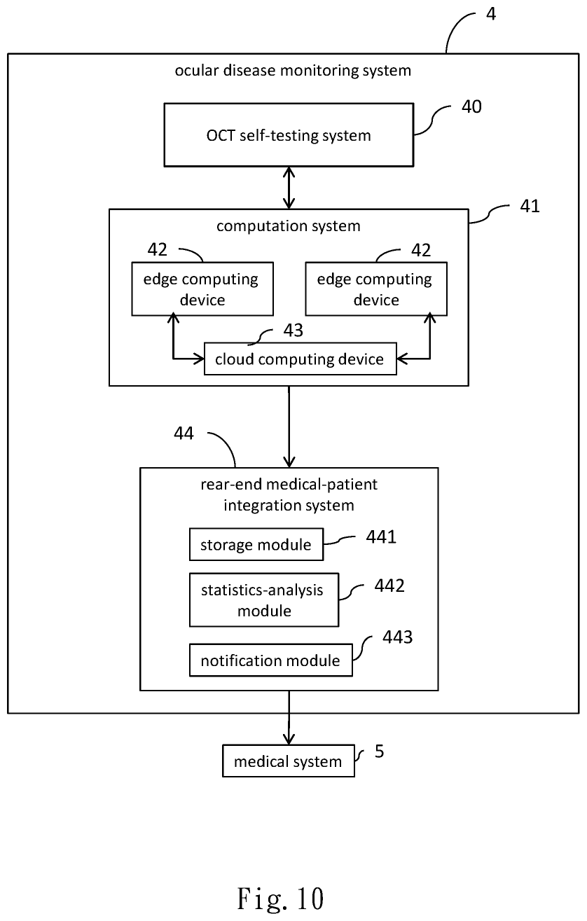

Refer to FIG. 10. FIG. 10 is a block diagram schematically showing an ocular disease monitoring system according to one embodiment of the present invention.

The ocular disease monitoring system 4 of the present invention comprises an OCT self-testing system 40 and a computation system 41.

The OCT self-testing system 40 is exactly the OCT self-testing system 1 shown in FIG. 1 and FIG. 2. The computation system 41 is in signal communication with the OCT self-testing system 40, receiving a plurality of ocular images, evaluating the plurality of ocular images to generate an evaluation result, and transmitting the evaluation result to the OCT self-testing system 40.

The computation system 41 includes a plurality of edge computing devices 42 and a cloud computing device 43. The plurality of edge computing devices 42 receives the plurality of ocular images to perform distributed-type computation and respectively generate distributed-type computation results. The plurality of edge computing devices 42 respectively transmits the distributed-type computation results to the cloud computing device 43. The cloud computing device 43 generates evaluation results according to the plurality of distributed-type computation results.

In another embodiment, the OCT self-testing system 1 may divide the plurality of ocular images into several groups according to the number of the plurality of edge computing devices 42. The plurality of edge computing devices 42 respectively calculates different groups of the plurality of ocular images to generate the distributed-type computation results.

In another embodiment, the OCT self-testing system 1 may divide the plurality of ocular images in a way: respectively allocating different pieces of ocular images to the edge computing devices 42 or allocating the same pieces of ocular images to the edge computing devices 42.

The cloud computing device 43 may include an artificial intelligence computing module (not shown in the drawing). The cloud computing device 43 receives the plurality of ocular images transmitted by the plurality of edge computing devices 42. The cloud computing device 43 uses the artificial intelligence computing module to evaluate the plurality of ocular images and generate evaluation results and then transmits the evaluation results to the edge computing devices 42. The edge computing devices 42 further transmit the evaluation results to the OCT self-testing system 40.

The artificial intelligence computing module may be trained via comparing pathological ocular images and normal ocular images, and the training data are stored thereinside. While receiving the plurality of ocular images from the edge computing devices 42, the artificial intelligence computing module can immediately determine the pathological states of the eyes.

The edge computing device 42 may be a terminal device that the user can use conveniently, such as a handheld device, a smart phone, a tablet computer, or a wearable device. The OCT self-testing system 40 is connected with the edge computing device 42 through a wireless technology, which may be but is not limited to be a WIFI technology or a Bluetooth technology. The edge computing device 42 exchanges data with the cloud computing device 43 through a mobile communication technology, which may be but is not limited to be a 4G technology or a 5G technology, functioning as a communication bridge between the OCT self-testing system 40 and cloud computing device 43.

The ocular disease monitoring system 4 of the present invention further comprises a rear-end medical-patient integration system 44, which includes a storage module 441, a statistics-analysis module 442, and a notification module 443. The storage module 441 stores the evaluation results fed back by the edge computing devices 42, whereby to facilitate disease tracking in future. The statistics-analysis module 442 performs statistics of the evaluation results and organizes them to generate a form. The notification module 443 transmits the form to a medical systems through a wired or wireless network. The medical system 5 may be an information system of a hospital or a mobile device of a clinic physician, such as a personal computer or a handheld device.

The testee may uses his personal handheld device to transmit the retinal images captured by the OCT self-testing system 40 to the cloud computing device 43. The cloud computing device 43 includes a pre-trained artificial intelligence computing module 431, which can determine the probability that the images involve pathological signs. Then, the cloud computing device 43 returns the results to the OCT self-testing system 40 through the handheld device, and the rear-end medical-patient integration system 44 records the results as a backup.

The evaluation result may involve the features and extent of the retinal disease. The cloud computing device 43 or the rear-end medical-patient integration system 44 may be used to generate a form of the evaluation results. Via referring to the trend presented by the form and the measurement records of the patient, the medical personnel may make a treatment plan for the patient. Therefore, the present invention may function as an information bridge between a patient of a retinal disease and a physician. Accordingly, the present invention can provide a home care service for the patients of retinal diseases and make the retinal diseases be diagnosed early and treated early. Then is efficiently saved medical resource and medical manpower.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. An optical coherence tomography self-testing system, comprising
a camera device, including
an image-capturing module, capturing a plurality of ocular images; and
a processing module, connected with the image-capturing module, determining whether a position offset value between a center of a pupil of a tested eyeball and an optical axis of the image-capturing module is within a preset error range, wherein if the position offset value is within the preset error range, the processing module determines whether the position offset value is unchanged within a first preset time interval; if the position offset value is unchanged within the first preset time interval, the processing module stores the plurality of ocular images, which is captured within the first time interval, as a plurality of displayed images;
an external display module, coupled to the processing module, displaying one of the plurality of displayed images and a status light after the processing module has completed image capturing, wherein the status light indicates a status of the image capturing performed by the image-capturing module; and
a communication module, connected with the processing module, and transmitting the plurality of ocular images to exterior.

2. The optical coherence tomography self-testing system according to claim 1, wherein according to a preset tracking rule and a preset focusing rule, the processing module analyzes a relative position of the optical axis O of the image-capturing module and the center of the pupil of the tested eyeball to generate the plurality of displayed images.

3. The optical coherence tomography self-testing system according to claim 2, wherein the image-capturing module includes
a first lens assembly, having a first-lens first side, and a first-lens second side, which are opposite to each other, wherein the first-lens first side faces the tested eyeball;
a second lens assembly, having a second-lens first side, and a second-lens second side, which are opposite to each other, wherein the second-lens first side faces the first-lens second side; the second lens assembly is disposed coaxially with the first lens assembly at the first-lens second side; the second lens assembly includes at least one liquid lens;
an illumination element, disposed at the first-lens second side, generating a light beam to illuminate an external region of the tested eyeball, wherein the light beam is focused at a fundus of the tested eyeball by the first lens assembly;
a splitter, disposed at a position between the first-lens second side and the second-lens first side, splitting the optical axis, which passes through the first lens assembly, into a first optical path and a second optical path, wherein the first optical path is an extension of the optical axis of the first lens assembly;
a sensing module, connected with the processing module, and disposed at the second-lens second side, wherein an imaging light beam of the tested eyeball is focused by the first lens assembly and the second lens assembly to form images on the sensing module; the sensing module receives the imaging light to form the plurality of ocular images;
a third lens assembly, coaxially disposed at the second optical path, having a third-lens first side and a third-lens second side, which are opposite to each other, wherein the third-lens first side faces the splitter;
an internal display module, connected with the processing module, and disposed at the third-lens second side, wherein the processing module transmits the plurality of displayed images to the internal display module; the internal display module presents the plurality of displayed images each including a picture frame of an area of a captured image; the internal display module generates an imaging light, which is corresponding to the plurality of displayed images, passes through the third lens assembly, the splitter and the first lens assembly in sequence, and is focused to the tested eyeball;
a first focal-length regulator, coupled to the processing module, driving the second lens assembly to move along the first optical path, adjusting a curvature of the at least one liquid lens to modify a focal length of the at least one liquid lens; and
a second focal-length regulator, coupled to the processing module, driving the internal display module to move along the second optical path of the third lens assembly, or adjusting a position of the third lens assembly, to make the imaging light form images on the fundus of the tested eyeball.

4. The optical coherence tomography self-testing system according to claim 2, wherein the preset tracking rule includes performing a pre-treatment of the plurality of ocular images to generate a binary image;
finding out a plurality of pupil-boundary characteristics from the binary image to obtain a pupil boundary; and
using a boundary fitting method to obtain the boundary of the contour of the pupil, and finding out coordinates of a pupil center.

5. The optical coherence tomography self-testing system according to claim 4, wherein the processing module stores positions of the plurality of pupil boundary characteristics in form of 2D coordinates; the processing module uses the coordinates of the pupil center to search outwards to find a center of a smallest circle surrounding the pupil center as a reference point; the processing module calculates a variance of the distances between the reference point and the plurality of pupil boundary characteristics to obtain the characteristics of the pupil.

6. The optical coherence tomography self-testing system according to claim 5, wherein the pre-treatment includes using the processing module to reduce size of the plurality of ocular images; eliminating noise signals from the plurality of ocular images; using an image-enhancing algorithm to output boundary-enhancing signals of the plurality of ocular images in a binary method; using the image-processing module to detect whether small-area noise signals, which make the boundary discontinuous, appear in the plurality of ocular images; if the small-area noise signals, which make the boundary discontinuous, appear in the plurality of ocular images, amending the small-area noise signals, which make the boundary discontinuous, in a morphological method to restore a portion of the ocular images and generate binary images.

7. The optical coherence tomography self-testing system according to claim 3, wherein the processing module analyzes the plurality of ocular images according to the preset focusing rule to obtain a signal-to-noise ratio of each of the ocular images, evaluates the signal-to-noise ratio according to a verification rule, and then controls the first focal-length regulator and the second focal-length regulator to adjust the focal length.

8. The optical coherence tomography self-testing system according to claim 7, wherein the preset focusing rule includes
using a central position of the ocular image to generate a window frame having a first preset size;
calculating gray-level histogram values inside the window frame, which range from 0 to 255;
setting a preset ratio, and calculating the pixels of the window frame and the preset ratio to obtain a dynamic threshold;
making the pixels inside the window frame, which are smaller than the dynamic threshold, be zero while an accumulated number of the gray-level histogram values is greater than the dynamic threshold;
taking an average of non-zero pixels remaining inside the window frame as a signal source;
taking two window frames having a second preset size respectively from a topmost area and a bottommost area of the ocular image, and taking an average of the window frames having the second preset size as a noise source; and
working out a value of the signal-to-noise ratio according to the signal source and the noise source.

9. The optical coherence tomography self-testing system according to claim 7, wherein the verification rule includes
storing the signal-to-noise ratios of the plurality of ocular images to a verification numeral group;
verifying whether the current signal-to-noise ratio is the largest one in the verification numeral group;
if the current signal-to-noise ratio is not the largest one in the verification numeral group, controlling the first focal-length regulator and the second focal-length regulator once again to make the imaging light focused on the sensing module to generate a refocused ocular image; and
if the current signal-to-noise ratio is the largest one in the verification numeral group, storing the current ocular image.

10. An optical coherence tomography method, which is applied to an optical coherence tomography self-testing system, wherein the optical coherence tomography self-testing system comprises a camera device, an external display module, and a communication module, wherein the camera device includes an image-capturing module and a processing module, wherein the optical coherence tomography method uses the optical coherence tomography self-testing system to undertake steps:
using the processing module to determine whether a position offset value between a center of a pupil of a tested eyeball and an optical axis of the image-capturing module is within a preset error range; if the position offset value is within the preset error range, using the processing module to determine whether the position offset value is unchanged within a first preset time interval; if the position offset value is unchanged within the first preset time interval, using the image-capturing module to capture a plurality of ocular images and storing the plurality of ocular images, which is captured within the first time interval, as a plurality of displayed images;
after the image-capturing module has completed image capturing, using the external display module to display one of the plurality of displayed images and a status light; and
using the communication module to transmit the plurality of ocular images to exterior.

11. The optical coherence tomography method according to claim 10, wherein the image-capturing module includes an internal display module, wherein the optical coherence tomography method further comprises steps:
according to a preset tracking rule and a preset focusing rule, using the processing module to analyze a relative position of the optical axis of the image-capturing module and the center of the pupil of the tested eyeball to generate the plurality of displayed images;
using the processing module to transmit the plurality of displayed images to the internal display module, and
using the internal display module to present the plurality of displayed images each including a picture frame of an area of the captured image.

12. The optical coherence tomography method according to claim 11, wherein using the processing module to analyze the plurality of ocular images according to the preset tracking rule further includes steps:
performing a pre-treatment of the plurality of ocular images to generate a binary image;
finding out a plurality of pupil-boundary characteristics from the binary image to obtain a pupil boundary; and
using a boundary fitting method to obtain the boundary of a contour of the pupil, and finding out coordinates of the center of the pupil.

13. The optical coherence tomography method according to claim 12, wherein finding out a plurality of pupil-boundary characteristics from the binary image further includes steps:
using the processing module to store positions of the plurality of pupil boundary characteristics in form of 2D coordinates;
using coordinates of a pupil center to search outwards to find a center of a smallest circle surrounding the pupil center as a reference point; and
calculating the variance of distances between the reference point and a plurality of pupil boundary characteristics to obtain characteristics of the pupil.

14. The optical coherence tomography method according to claim 12, wherein the pre-treatment includes further includes steps:
using the processing module to reduce size of the plurality of ocular images;
eliminating noise signals from the plurality of ocular images;
using an image-enhancing algorithm to output boundary-enhancing signals of the plurality of ocular images in a binary method; and
using the image-processing module to detect whether small-area noise signals, which make the boundary discontinuous, appear in the plurality of ocular images; if the small-area noise signals, which make the boundary discontinuous, appear in the plurality of ocular images, amending the small-area noise signals, which make the boundary discontinuous, in a morphological method to restore a portion of the ocular images and generate binary images.

15. The optical coherence tomography method according to claim 11, wherein the processing module analyzes the plurality of ocular images according to the preset focusing rule to obtain a signal-to-noise ratio of each of the ocular images, evaluates the signal-to-noise ratio according to a verification rule, and then controls a first focal-length regulator and a second focal-length regulator to adjust a focal length.

16. The optical coherence tomography method according to claim 15, wherein analyzing the plurality of ocular images according to the preset focusing rule further including steps:
using a central position of the ocular image to generate a window frame having a first preset size;
calculating gray-level histogram values inside the window frame, which range from 0 to 255;
setting a preset ratio, and calculating the pixels of the window frame and the preset ratio to obtain a dynamic threshold;
making the pixels inside the window frame, which are smaller than the dynamic threshold, be zero while an accumulated number of the gray-level histogram values is greater than the dynamic threshold;
taking an average of non-zero pixels remaining inside the window frame as a signal source;
taking two window frames having a second preset size respectively from a topmost area and a bottommost area of the ocular image, and taking an average of the window frames having the second preset size as a noise source; and
working out a value of the signal-to-noise ratio according to the signal source and the noise source.

17. The optical coherence tomography method according to claim 15, wherein evaluating the signal-to-noise ratio according to a verification rule further includes steps:
storing the signal-to-noise ratios of the plurality of ocular images to a verification numeral group;
verifying whether the current signal-to-noise ratio is the largest one in the verification numeral group;
if the current signal-to-noise ratio is not the largest one in the verification numeral group, controlling the first focal-length regulator and the second focal-length regulator once again to make the imaging light focused on the sensing module to generate a refocused ocular image; and
if the current signal-to-noise ratio is the largest one in the verification numeral group, storing the current ocular image.

18. An ocular disease monitoring system, comprising
an optical coherence tomography self-testing system, further comprising
a camera device, including
an image-capturing module, capturing a plurality of ocular images; and
a processing module, connected with the image-capturing module, determining whether a position offset value between a center of a pupil of a tested eyeball and an optical axis of the image-capturing module is within a preset error range, wherein if the position offset value is within the preset error range, the processing module determines whether the position offset value is unchanged within a first preset time interval; if the position offset value is unchanged within the first preset time interval, the processing module stores the plurality of ocular images, which is captured within the first time interval, as a plurality of displayed images;
an external display module, coupled to the processing module, displaying one of the plurality of displayed images and a status light after the processing module has completed image capturing, wherein the status light indicates a status of the image capturing performed by the image-capturing module; and
a communication module, connected with the processing module, and transmitting the plurality of ocular images to exterior;
a computation system, in signal communication with the optical coherence tomography self-testing system, receiving a plurality of ocular images, evaluating the plurality of ocular images to generate an evaluation result, and transmitting the evaluation result to the optical coherence tomography self-testing system.

19. The ocular disease monitoring system according to claim 18, wherein the communication module transmits the plurality of ocular images to the computation system; the computation system includes a plurality of edge computing devices and a cloud computing device; the plurality of edge computing devices receives the plurality of ocular images to perform distributed-type computation and respectively generate distributed-type computation results; the plurality of edge computing devices respectively transmits the distributed-type computation results to the cloud computing device; the cloud computing device generates evaluation results according to the plurality of distributed-type computation results

20. The ocular disease monitoring system according to claim 18, further comprising a rear-end medical-patient integration system, wherein the rear-end medical-patient integration system includes
a storage module, storing the evaluation results fed back by the computation system;
a statistics-analysis module, performing statistics of the evaluation results and recognizing the evaluation results to generate a form; and
a notification module, transmitting the form to a medical system.
